(19) United States
(12) Patent Application Publication
(10) Pub. No.: US 2011/0124978 A1
(43) Pub. Date: May 26, 2011

(54) HEALTH AND FITNESS SYSTEM

(76) Inventor: MICHAEL C. WILLIAMS, Santa Barbara, CA (US)

(21) Appl. No.: 12/625,386

(22) Filed: Nov. 24, 2009

(51) Int. Cl. A61B 5/00; G06Q 30/00; G06Q 50/00; G06F 3/01; H04B 1/38; G06F 3/048

(52) U.S. Cl. 600/301; 715/760; 455/90.1; 715/764; 705/26.7

(57) ABSTRACT

The present invention provides a health and fitness system, comprising a means for providing access to the health and fitness application, a means for retrieving data from a health and fitness data source, a means for selectively integrating health and fitness data among multiple health and fitness applications, sensors, and devices, a means for logging health and fitness data, a means for purchasing a health and fitness item from a catalog of items, a means for sharing health and fitness data with a remote computing device, and a means for messaging to and from a smart phone.

### FIELD OF THE INVENTION

The present invention relates generally to health and fitness, and more particularly, some embodiments relate to a health and fitness system that aggregates, tracks and manages health and fitness data.

### DESCRIPTION OF THE RELATED ART

Advancements in technology have changed the way people approach health and fitness. Over time, useful tools and devices have been developed that provide useful information in attaining and maintaining health and fitness goals. In addition to tracking one's goals, various tools and devices provide real-time information on performance, gauge one's performance to others, and offer tips and suggestions in one's diet and exercise routines. Tools and devices such as heart rate monitors, pace monitors or pedometers, cycling computers, glucose meters, weight scales, blood pressure cuffs, physical training machines integrated with sensors, and other health and fitness sensor devices are commonplace in the world of fitness.

Maintaining a record of such health and fitness data can be useful in planning and assessing progress regarding physical condition. For example, trends in heart rate, weight, distance, power, speed, calories burned during exercise, cadence, and the other measurements that these devices provide can be helpful in planning future exercise workouts or regimens. Often these systems are equipped with various communications protocols and software methods to allow data transfer from the device to a central database or website.

As such, several of the fitness tools and devices employ interfaces that allow data to be transferred off the device, and stored and/or analyzed for performance purposes. For example, a fitness computer can monitor heart rate and caloric expenditure. Such a fitness computer could then interface with a computer, through a wired or wireless connection, allowing it to store and track various fitness performance indicators, such as historical calorie consumption or average heart rate.

Additionally, the tools and devices are not limited just to exercise. It is commonly understood that one's diet is just as important to achieving and maintaining health and fitness goals as exercise. To that effect, tools and devices have been developed dealing with dieting and eating right. For example, healthy eating applications have been created to offer both advice and recipes for healthy food.

Unfortunately, the tools and devices described here and their like exist and operate in an individualistic manner. Often, the tools and devices are implemented as separate systems that lack compatibility with each other due to differences in methods, functionality, and/or data format. As such, it is exceedingly difficult to aggregate data from these tools and devices. This lack of interoperability not only influences a user's decision on future purchases of such tools and devices, but also serves as an obstacle to widespread use.

### BRIEF SUMMARY OF EMBODIMENTS OF THE INVENTION

According to various embodiments of the invention, systems and methods that aggregate, track and manage health and fitness data are provided. The health and fitness data for these systems and methods can originate from a variety of sources, including health monitoring devices. In addition, various embodiments provide a system and method for aggregating together a variety of disparate and/or incompatible health and fitness systems and applications into one integrated health and fitness system. In further embodiments, the resulting integrated system may be modular in nature, thereby allowing a user to selectively add and remove the health and fitness systems and applications from the integrated system based on user preferences.

According to one embodiment of the invention, a health and fitness system is provided, comprising: a means for providing access to a health and fitness application; a means for retrieving data from a health and fitness data source; a means for selectively integrating health and fitness data among multiple health and fitness applications, sensors, and devices; a means for logging health and fitness data; a means for purchasing a health and fitness item from a catalog of items; a means for sharing health and fitness data with a remote computing device; and a means for messaging to and from a smart phone. In some embodiments, the means for sharing health and fitness data includes interacting with a personal trainer website, a medical website, or medical practice management software.

In some embodiments, the health and fitness application is designed for different modalities of health and fitness. Accordingly, each health and fitness application may be designed for a specific fitness modality that determines which parameters or metrics of interest are displayed or recorded for the fitness activity, and in what format such parameters or metrics are displayed. For example, there may be a specific health and fitness application for running on a treadmill, where the application is designed to interface with the treadmill computer to retrieve metrics data, and the application displays and records such metrics as cadence, time elapsed in minutes, and heart rate as the user runs on the treadmill. In addition, the parameters, metrics, and format, may change from application to application. For example, an application for hiking may display and record the route taken based on GPS coordinates, cadence, and time elapsed. An application for weight training, on the other hand, will display and record the number of sets, the number of repetitions per a set, and the weight per a set.

In further embodiments, the means for retrieving data from a health and fitness data source involves pulling data from a health and fitness device, which serves as the data source. Examples of health and fitness devices include heart rate monitors, blood pressure monitors, glucose meters, cycling sensors, cycling computers, rowing sensors, rowing computers, treadmill sensors, treadmill computers, elliptical trainer sensors, elliptical trainer computers, global-positioning system devices, running sensors, pedometers, scales, or fitness watches. Some embodiments may retrieve information from the health and fitness device and either upload it to a remote computing device, or store it locally within the system. Additionally, for some embodiments, the device is a mobile device configured to interact with a health monitoring device.

In some embodiments, the health and fitness items that can be purchased include health monitoring equipment and health and fitness contents, which can be physical or digital. For certain embodiments, when the content purchased is digital, the embodiment can immediately receive the digital health and fitness content and store it in the health and fitness system.

In certain embodiments, the means for logging health and fitness data provides a personal health diary, a weight scale log, caloric intake, a blood pressure log, or glucose log. Depending on the embodiment, these diaries and logs may receive health and fitness data either through manual or automatic means.

In additional embodiments, the remote computing device to which the system communicates is on the Internet. In some such embodiments, the remote computing device may be a website or simply a server that can send and receive health and fitness information. For example, the remote computing device may be a health and fitness website where a user stores, tracks, and manages their health and fitness information, wherein a health and fitness system in accordance with an embodiment could access and utilize such information.

In further embodiments, the message provided by the system may be a positive psychological message used to encourage the user in their health and fitness endeavors. Additional messages can include suggestions, tips and advice relating to health and/or fitness.

In other embodiments, the system further comprises a graphical user interface that interfaces with at least one of the means that is part of the system. For example, the graphical user interface may be configured to interface with the means for providing access to a health and fitness application. In another example, the graphical user interface may be configured to interface with a means for logging health and fitness data.

According to further embodiments, various systems, methods, and operations described above are implemented using a computer. For example, some embodiments provide for a computer program product comprising a computer useable medium having computer program code embodied therein, configured to cause a computing device to operate in accordance with aspects of the invention as described herein.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

FIG. 1 is a diagram illustrating an example system for retrieval, storage and management of health and fitness data in accordance with an embodiment of the invention.

FIG. 2 is a diagram illustrating an example health and fitness system in accordance with an embodiment of the invention.

FIG. 3 is a diagram illustrating an example computing module for implementing various embodiments of the invention.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF THE EMBODIMENTS OF THE INVENTION

The present invention is directed toward a system and method for managing health and fitness related data. In some embodiments, a communications module interfaces with a mobile communications device to provide a bridging system between a variety of health and fitness data generating devices and a centralized data server.

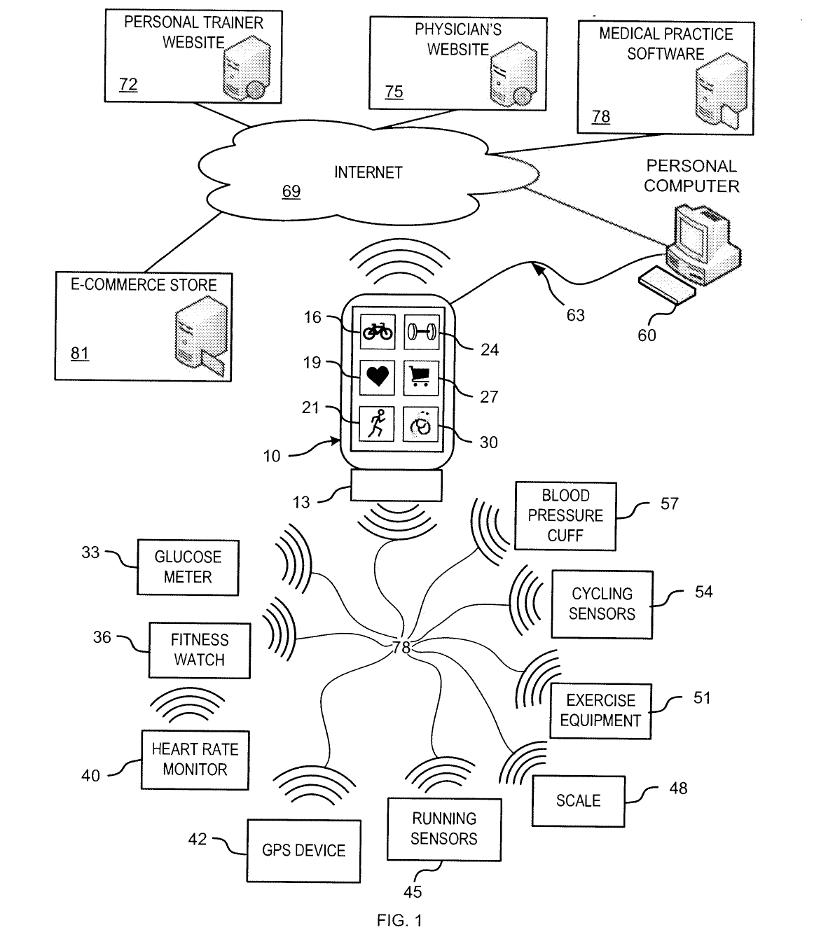

FIG. 1 is a diagram illustrating an example system of health and data retrieval, storage and management in accordance with an embodiment of the invention. Illustrated is a mobile computing device 10 equipped with a communications interface 13 that allows the device 10 to communicate with various health devices (33 through 57). In the illustrated embodiment, the device 10 is operating a health and fitness system. This system may also be referred to herein as a health and fitness ecosystem. Such an ecosystem is capable of integrating multiple health and fitness applications, and retrieving, storing and managing health and fitness data.

The mobile computing device 10 may be equipped with a communications interface 13 that allows the device 10 to communicate with various health monitoring devices. Additional communication interfaces (not shown) allow the device 10 to communicate with an external computer network, such as the Internet 69. Examples of a mobile computing device may comprise a mobile phone, such as a smart phone, or a PDA, that has a data connection to the Internet 69 and the ability to load and execute computer programs via an operating system. For example, computing device 10 may comprise a smart phone running an operating system and may be programmed with a health and fitness system application that allows it to communicate with the health monitoring devices (33 through 57) and to allow health and fitness data collected from the health monitoring devices to be uploaded or synchronized to an external source, such as a website.

It should be noted that different health monitoring devices may be configured to communicate according to various protocols. For example, a cyclometer might be configured to wirelessly communicate according to an ultra low power protocol such as the ANT+ protocol, while a GPS device might be configured to wirelessly communicate according to the Bluetooth protocol. Accordingly, the communications module 76 may be configured to communicate according to a plurality of different communications protocols to provide the obtained data to the mobile device 75.

In the illustrated example, the health and fitness system operating on device 10 comprises applications that help store, track and manage data provided by specific types of fitness activities. Here the applications are a cycling application 16, a weight training application 24, a cardio application 19, and a running application 21. Other applications relating to specific fitness activities could include, without limitation, a yoga application, a hiking application, a Spinning® application, a Pilates application, a rowing application, and a walking application.

The illustrated system further comprises a store 27, which may allow a user to purchase health and fitness equipment, such as heart monitoring sensors, or health or fitness content such as a workout routine. Additionally, the system comprises a medical application 30, which can provide the user with health and medical information or advice. After reading the description provided herein, it will be apparent to one of ordinary skill in the art that a variety of applications relating to health and fitness could operate on health and fitness systems in accordance with the invention. Additionally, depending on the embodiment, the application may retrieve and store information locally on the mobile device 10, or on a remote computing device connected to the mobile computing device 10 through a network connection. For example, the store 27 may function by interfacing with an e-commerce store 81 via the Internet 69.

Through the illustrated system, a user may have access to a variety of health monitoring devices. For example, an athlete such as a runner may be equipped with a variety of data collecting devices during an exercise period, such as a running sensor 45, a GPS device 42, a heart rate monitor 40, and a fitness watch 39. Various devices may be configured to temporarily store data during the exercise period and then provide the system access to this data at a later point in time. For example, GPS device 42 may be configured to store a route taken by a runner during a run, and then to provide that route to the health fitness system at a later point in time. Furthermore, some health monitoring devices may be pre-configured to interoperate with each other before ultimate collection by the health and fitness system. For example, a fitness watch 36 may be configured to receive and collect data during an exercise period from other health monitoring devices, such as heart rate monitor 40.

Other health and fitness devices that can provide the system with data include glucose meter 33 and blood pressure cuff 57, both of which provide readings on body vitals, and a scale 48, which can provide the system with a user's weight. Additional exercise equipment 51 may include sensors that supply health and information data to the illustrated system.

The illustrated system is further configured to interface with a personal trainer website 75, which provide the user of the mobile device 10 with personal fitness training via the health and fitness system, and a physician's websites 75 through which the mobile device 10 may provide health and fitness information to the user's physician in almost real-time. Optionally, the system may be configured to interface with the medical practice software 78 providing an alternative means for presenting health and fitness information to the user's physician. The system may synchronize or upload health and system data with the user's personal computer 60, which may be facilitated through a direct connection 63 with the personal computer or through an Internet connection. The direct connection 63 may be a wired or wireless connection between the personal computer 60 and the mobile device 10.

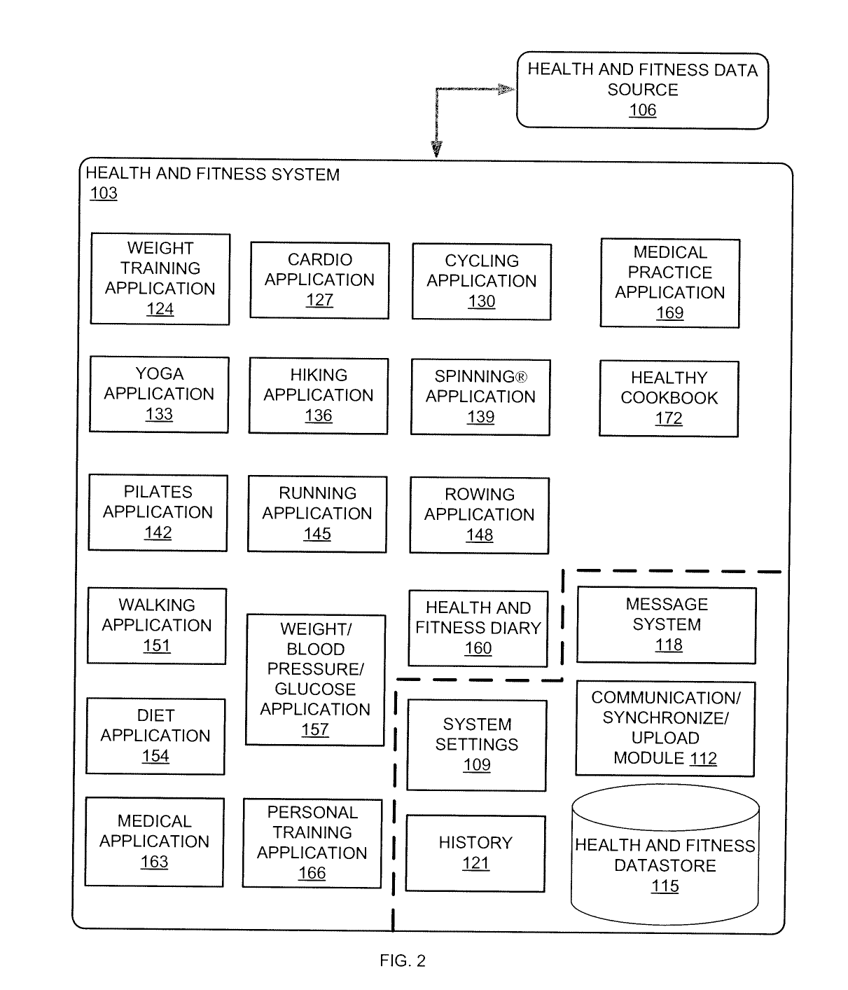

FIG. 2 is a diagram illustrating an example health and fitness system 103 in accordance with an embodiment of the invention. Health and fitness system 104 may operate on a mobile device similar to that illustrated in FIG. 1. As illustrated, system 103 interfaces with a health and fitness data source 106, which supplies system 103 with health and fitness data. Examples of health and fitness data sources can include health monitoring devices, like those described earlier, or remote computing devices that are operating a website or a server and present on a server. The system 103 may utilize the information retrieved directly, or store the information locally on a health and fitness datastore 115.

The illustrated system 103 is configured with various fitness activity applications, including weight training application 124, cardio application 127, cycling application 130, yoga application 133, hiking application 136, Spinning® application 139, Pilates application 142, running application 145, rowing application 148, and walking application 151. To assist the user in their diet goals, the system 103 includes a diet application 163 that can track such things as caloric intake, and a health and healthy cookbook that can provide recipes for healthy eating. The system 103 also supports an application 157 that tracks a user's vitals, such as weight, blood pressure, and glucose levels.

The system 103 may be configured to route health and fitness data from the data source 106 to applications operating on the system, or health and fitness data between applications. For example, a GPS device may provide data on the route taken by a runner during a run, and a heart rate monitor and fitness watch may provide data on vitals during the run. This information could be routed by the system 103 to the running application 145, or to the health and fitness datastore 115 for use at a later point in time.

With regard to interfacing with external systems, the system 103 is additionally configured with a medical application 163 for accessing medical information, a personal training application 166, and a medical practice application 169 for sending and receiving health information to the user's doctor. Health and fitness diary 160 provides a user with a means for a user to maintain a personal log on their health and fitness goals and progress.

As illustrated, system 103 is further implemented using various modules that provide it with additional functionality. For example, a message system 118 allows system 103 to provide a user with positive psychological messages designed to encourage a user in their health and fitness goals. The message system 118 may also provide advice and suggestions based on health and fitness data that the system 103 has obtained. System settings module 109 maintains settings for the system 103, such as which applications in the system 103 a user desired to utilize and how the applications access health and fitness data. The history module 121 may provide such functions as a history of applications used, or a history of health or fitness activities. The communication, synchronization, and upload module 112 may enable system 103 to access health and fitness data sources 106, or interface personal training or physicians websites.

As used herein, the term set may refer to any collection of elements, whether finite or infinite. The term subset may refer to any collection of elements, wherein the elements are taken from a parent set; a subset may be the entire parent set. The term proper subset refers to a subset containing fewer elements than the parent set. The term sequence may refer to an ordered set or subset. The terms less than, less than or equal to, greater than, and greater than or equal to, may be used herein to describe the relations between various objects or members of ordered sets or sequences; these terms will be understood to refer to any appropriate ordering relation applicable to the objects being ordered.

The term tool can be used to refer to any apparatus configured to perform a recited function. For example, tools can include a collection of one or more modules and can also be comprised of hardware, software or a combination thereof. Thus, for example, a tool can be a collection of one or more software modules, hardware modules, software/hardware modules or any combination or permutation thereof. As another example, a tool can be a computing device or other appliance on which software runs or in which hardware is implemented.

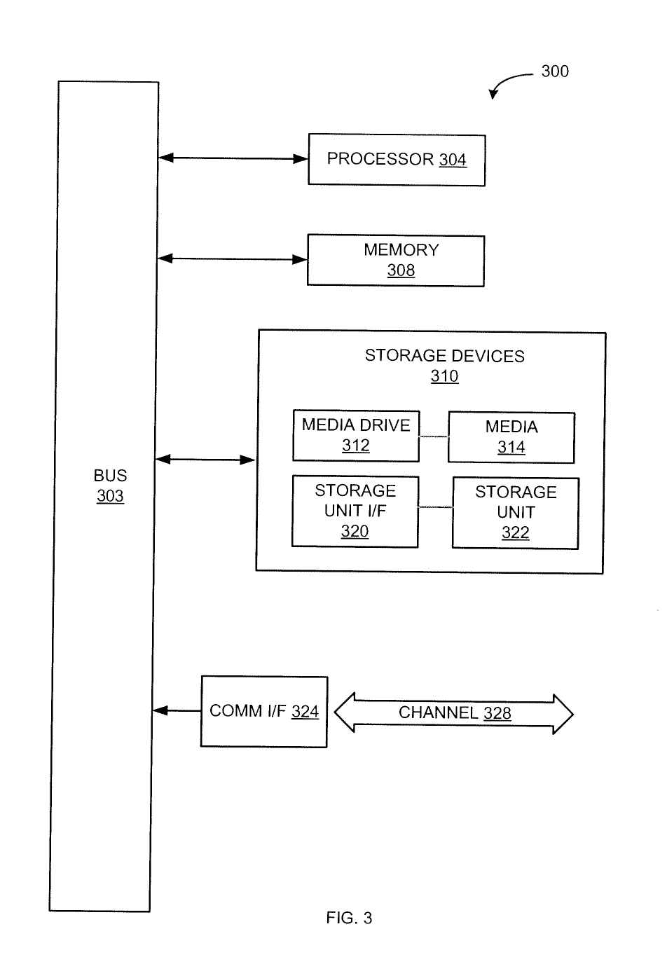

As used herein, the term module might describe a given unit of functionality that can be performed in accordance with one or more embodiments of the present invention. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the invention are implemented in whole or in part using software, in one embodiment, these software elements can be implemented to operate with a computing or processing module capable of carrying out the functionality described with respect thereto. One such example computing module is shown in FIG. 3. Various embodiments are described in terms of this example-computing module 300. After reading this description, it will become apparent to a person skilled in the relevant art how to implement the invention using other computing modules or architectures.

Referring now to FIG. 3, computing module 300 may represent, for example, computing or processing capabilities found within desktop, laptop and notebook computers; hand-held computing devices (PDA's, smart phones, cell phones, palmtops, etc.); mainframes, supercomputers, workstations or servers; or any other type of special-purpose or general-purpose computing devices as may be desirable or appropriate for a given application or environment. Computing module 300 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 300 might include, for example, one or more processors, controllers, control modules, or other processing devices, such as a processor 304. Processor 304 might be implemented using a general-purpose or special-purpose processing engine such as, for example, a microprocessor, controller, or other control logic. In the illustrated example, processor 304 is connected to a bus 302, although any communication medium can be used to facilitate interaction with other components of computing module 300 or to communicate externally.

Computing module 300 might also include one or more memory modules, simply referred to herein as main memory 308. For example, preferably random access memory (RAM) or other dynamic memory, might be used for storing information and instructions to be executed by processor 304. Main memory 308 might also be used for storing temporary variables or other intermediate information during execution of instructions to be executed by processor 304. Computing module 300 might likewise include a read only memory (“ROM”) or other static storage device coupled to bus 302 for storing static information and instructions for processor 304.

The computing module 300 might also include one or more various forms of information storage mechanism 310, which might include, for example, a media drive 312 and a storage unit interface 320. The media drive 312 might include a drive or other mechanism to support fixed or removable storage media 314. For example, a hard disk drive, a floppy disk drive, a magnetic tape drive, an optical disk drive, a CD or DVD drive (R or RW), or other removable or fixed media drive might be provided. Accordingly, storage media 314 might include, for example, a hard disk, a floppy disk, magnetic tape, cartridge, optical disk, a CD or DVD, or other fixed or removable medium that is read by, written to or accessed by media drive 312. As these examples illustrate, the storage media 314 can include a computer usable storage medium having stored therein computer software or data.

In alternative embodiments, information storage mechanism 310 might include other similar instrumentalities for allowing computer programs or other instructions or data to be loaded into computing module 300. Such instrumentalities might include, for example, a fixed or removable storage unit 322 and an interface 320. Examples of such storage units 322 and interfaces 320 can include a program cartridge and cartridge interface, a removable memory (for example, a flash memory or other removable memory module) and memory slot, a PCMCIA slot and card, and other fixed or removable storage units 322 and interfaces 320 that allow software and data to be transferred from the storage unit 322 to computing module 300.

Computing module 300 might also include a communications interface 324. Communications interface 324 might be used to allow software and data to be transferred between computing module 300 and external devices. Examples of communications interface 324 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 324 might typically be carried on signals, which can be electronic, electromagnetic (which includes optical) or other signals capable of being exchanged by a given communications interface 324. These signals might be provided to communications interface 324 via a channel 328. This channel 328 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, memory 308, storage unit 320, media 314, and channel 328. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing module 300 to perform features or functions of the present invention as discussed herein.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A health and fitness system, comprising:
a means for providing access to the health and fitness application;
a means for retrieving data from a health and fitness data source;
a means for selectively integrating health and fitness data among multiple health and fitness applications, sensors, and devices;
a means for logging health and fitness data;
a means for purchasing a health and fitness item from a catalog of items;
a means for sharing health and fitness data with a remote computing device; and
a means for messaging to and from a smart phone.

2. The health and fitness system of claim 1, wherein the means for sharing health and fitness data includes interacting with a personal trainer website, the medical website, or the practice management software.

3. The health and fitness system of claim 1, wherein the health and fitness data source is a health and fitness device.

4. The health and fitness system of claim 3, wherein the device is a heart rate monitor, a blood pressure monitor, a glucose meter, a cycling sensor, a cycling computer, a rowing sensor, a rowing computer, a treadmill sensor, a treadmill computer, an elliptical trainer sensor, an elliptical trainer computer, a global-positioning system device, a running sensor, a pedometer, a scale, or a fitness watch.

5. The health and fitness system of claim 3, wherein the device is a mobile device configured to interact with a health monitoring device.

6. The health and fitness system of claim 1, wherein the health and fitness data source is a website.

7. The health and fitness system of claim 1, wherein the health and fitness item includes health monitoring equipment and health and fitness content.

8. The health and fitness system of claim 1, wherein the means for logging health and fitness data provides a personal health diary, a weight scale log, caloric intake, a blood pressure log, or glucose log.

9. The health and fitness system of claim 1, wherein the remote computing device is on the Internet.

10. The health and fitness system of claim 1, wherein the message is a positive psychological message, or a suggestion relating to health or fitness.

11. The health and fitness system of claim 1, further comprising a graphical user interface that interfaces with at least one of the means.

12. A computer program product comprising a computer useable medium having computer program code embodied therein configured to cause a computing device to:
provide access a health and fitness application;
retrieve data from a health and fitness data source;
selectively integrate health and fitness data among multiple health and fitness applications, sensors, and devices;
log health and fitness data;
purchase a health and fitness item from a catalog of items;
communicate health and fitness data with a remote computing device; and
send messages to and receive messages from a smart phone.

13. The computer program product of claim 12, wherein the means for sharing health and fitness data includes interacting with a personal trainer website, the medical website, or the practice management software.

14. The computer program product of claim 12, wherein the health and fitness data source is a health and fitness device.

15. The computer program product of claim 14, wherein the device is a heart rate monitor, a cycling computer, a rowing computer, a treadmill computer, or an elliptical trainer computer.

16. The computer program product of claim 14, wherein the device is a mobile device configured to interact with a health monitoring device.

17. The computer program product of claim 12, wherein the health and fitness data source is a website.

18. The computer program product of claim 12, wherein the health and fitness item includes health monitoring equipment and health and fitness content.

19. The computer program product of claim 12, wherein the means for logging health and fitness data provides a personal health diary, a weight scale log, caloric intake. a blood pressure log, or glucose log.

20. The computer program product of claim 12, wherein the remote computing device is on the Internet.

21. The computer program product of claim 12, wherein the message is a positive psychological message, or a suggestion relating to health or fitness.

22. The computer program product of claim 11, configured to cause a computing device to provide a graphical user interface that provides access to at least one of the means.
